(19) United States
(12) Patent Application Publication
(10) Pub. No.: US 2023/0172866 A1
(43) Pub. Date: Jun. 8, 2023

(54) ORAL PHARMACEUTICAL COMPOSITION

(71) Applicant: SICHUAN HAISCO PHARMACEUTICAL CO., LTD., Chengdu, Sichuan (CN)

(72) Inventors: Ling FU, Chengdu, Sichuan (CN); Shunhong WANG, Chengdu, Sichuan (CN); Dong REN, Chengdu, Sichuan (CN); Xiaoping LI, Chengdu, Sichuan (CN); Yi MO, Chengdu, Sichuan (CN)

(21) Appl. No.: 17/906,555

(22) PCT Filed: Mar. 17, 2021
(86) PCT No.: PCT/CN2021/081224

§ 371 Date: Sep. 16, 2022

(30) Foreign Application Priority Data
Mar. 18, 2020 (CN) 202010179149.6
Mar. 12, 2021 (CN) 202110268405.3

(51) Int. Cl. A61K 9/48; A61K 38/07; A61K 9/20

(52) U.S. Cl. CPC A61K 9/4858; A61K 38/07; A61K 9/2013; A61K 9/4866; A61K 9/2054; A61K 9/2027

(57) ABSTRACT

An oral pharmaceutical composition of a peptide amide compound (compound A). The present invention further relates to a method for preparing an oral pharmaceutical composition, and the use of the oral pharmaceutical composition in preparing a drug for treating diseases or conditions related to the κ-opioid receptor.

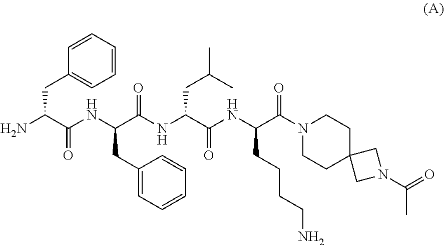 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

### TECHNICAL FIELD

The present invention relates to an oral pharmaceutical composition of a peptide amide compound. The present invention further relates to a method for preparing the oral pharmaceutical composition and the use thereof.

### BACKGROUND ART

Polypeptide drugs can be used for treating various diseases due to its wide indications, high safety, remarkable selectivity and effectiveness. With the development of biotechnology, a large number of peptide drugs have entered the market. Currently, the commercially available peptide preparations are mainly in the form of injections and nasal sprays, which bring great inconvenience to patients. For patients, the oral administration is simpler and does not directly damage the skin or mucous membranes, thereby reducing patients' pain and improving patient compliance. However, the oral administration of polypeptide drugs is limited by a series of barriers, specifically, the polypeptide drugs are easily degraded and destroyed by proteolytic enzymes in the digestive tract, and are difficult to pass through the epithelial cell layer of the inner wall of the small intestine due to the large molecular weight, resulting in low oral bioavailability and limiting the clinical use thereof. For example, the absolute bioavailability of the listed semaglutide tablets is only 0.4%-1%. In order to achieve the blood concentration required for the onset of action, the oral dose is relatively high, and it is difficult to achieve oral administration of some polypeptide drugs with a high cost or a narrow therapeutic window. Therefore, improving the oral bioavailability of polypeptide drugs is the key to achieving the oral administration of polypeptide drugs.

Most polypeptide drugs are susceptible to degradation by a protease activated by gastric acid, resulting in a decrease in the activity thereof. Enteric coating technology is usually used to protect polypeptides, which, however, requires complicated process and is of high cost.

WO 2019/015644 discloses a class of peptide amide compounds with novel structure and good analgesic effect, and the general formula thereof is as follows:

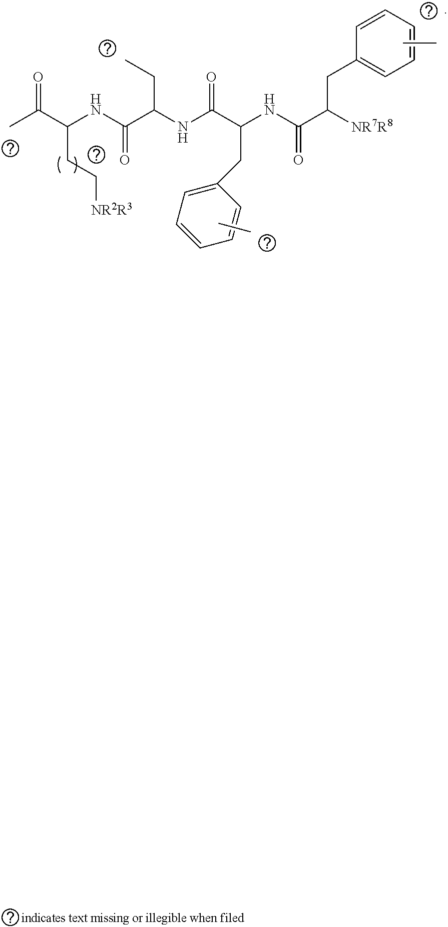

Such compounds have a significant agonizing effect on human κ-opioid receptors. Specifically, compound A is disclosed therein:

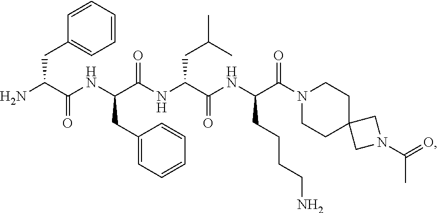 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

with a chemical name of 7-(D-phenylalanyl-D-phenylalanyl-D-leucyl-D-lysyl)-2-acetyl-2,7-diazaspiro[3.5]nonane.

### SUMMARY OF THE INVENTION

The present invention provides an oral polypeptide pharmaceutical composition, which is prepared by combining a polypeptide drug with a specific absorption enhancer and other non-active ingredients, thereby promoting the permeability of the polypeptide drug in the gastrointestinal tract, improving the oral bioavailability of the polypeptide drug, and achieving the oral administration so as to improve patient compliance.

The present invention provides a simple intragastric administration technology, which greatly simplifies the technological process and can greatly improve the oral bioavailability of the polypeptide compound A.

One object of the present invention is to provide an oral pharmaceutical composition of compound A:

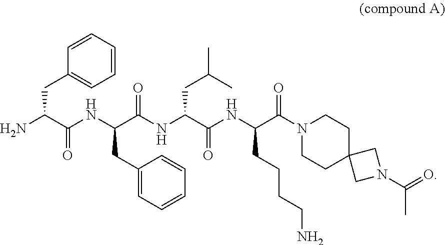 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

The present invention significantly improves the oral bioavailability of compound A, and is more favorable to develop a drug with the compound A.

Another object of the present invention is to provide a method for preparing the oral pharmaceutical composition.

Another object of the present invention is to provide the use of the oral pharmaceutical composition.

In one aspect, the present invention provides an oral pharmaceutical composition comprising:

a) compound A:

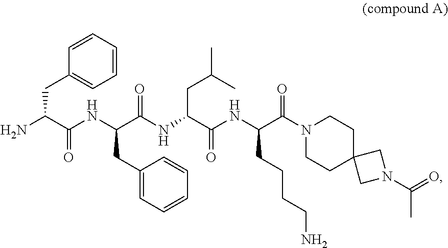 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer.

In some embodiments of the present invention, the absorption enhancer is selected from one or more of N-[8-(2-hydroxybenzoyl)amino]octanoic acid or a pharmaceutically acceptable salt thereof, 4-[(4-chloro-2-hydroxy-benzoyl)amino]butyric acid or a pharmaceutically acceptable salt thereof, lauroyl-L-carnitine or a hydrochloride thereof, sodium caprylate, sodium caprate, capric acid, caprylocaproyl macrogolglyceride; preferably, the pharmaceutically acceptable salt is selected from sodium salt, potassium salt or calcium salt.

In some embodiments of the present invention, the oral pharmaceutical composition comprises:

a) compound A:

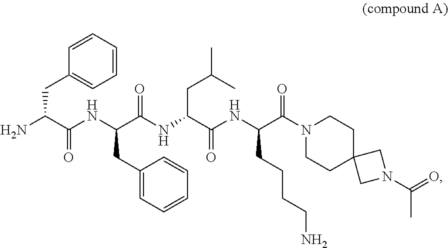 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from N-[8-(2-hydroxybenzoyl)amino]octanoic acid or a pharmaceutically acceptable salt thereof.

In some embodiments of the present invention, the weight ratio of compound A to N-[8-(2-hydroxybenzoyl)amino]octanoic acid or a pharmaceutically acceptable salt thereof is 1:20-1:80, preferably 1:40-1:60, further preferably 1:40.

In some embodiments of the present invention, the weight ratio of compound A to N-[8-(2-hydroxybenzoyl)amino]octanoic acid or a pharmaceutically acceptable salt thereof is 1:20.

In some embodiments of the present invention, the weight ratio of compound A to N-[8-(2-hydroxybenzoyl)amino]octanoic acid or a pharmaceutically acceptable salt thereof is 1:30.

In some embodiments of the present invention, the weight ratio of compound A to N-[8-(2-hydroxybenzoyl)amino]octanoic acid or a pharmaceutically acceptable salt thereof is 1:40.

In some embodiments of the present invention, the weight ratio of compound A to N-[8-(2-hydroxybenzoyl)amino]octanoic acid or a pharmaceutically acceptable salt thereof is 1:80.

In some embodiments of the present invention, the absorption enhancer is N-[8-(2-hydroxybenzoyl)amino]sodium caprylate.

In some embodiments of the present invention, the absorption enhancer is N-[8-(2-hydroxybenzoyl)amino]potassium caprylate.

In some embodiments of the present invention, the absorption enhancer is N-[8-(2-hydroxybenzoyl)amino]calcium caprylate.

In some embodiments of the present invention, the oral pharmaceutical composition comprises:

a) compound A:

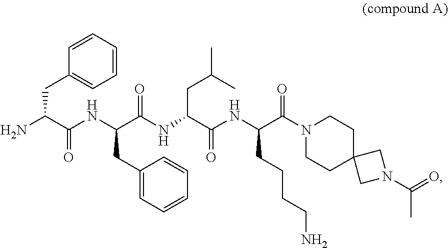 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from N-[8-(2-hydroxybenzoyl)amino]octanoic acid or a pharmaceutically acceptable salt thereof;
c) one or more non-active ingredients selected from:
(1) a filler, which is preferably selected from one or more of microcrystalline cellulose, lactose, mannitol, anhydrous calcium hydrogenphosphate, pregelatinized starch, calcium hydrogenphosphate dihydrate;
(2) a binder, which is preferably selected from one or more of povidone, copovidone, a cellulose derivative, wherein the cellulose derivative is selected from one or more of hydroxypropylcellulose, hypromellose, methyl cellulose;
(3) a disintegrant, which is preferably selected from one or more of crospovidone, low substituted hydroxypropyl cellulose, croscarmellose sodium, sodium carboxymethyl starch;
(4) a pH regulator, which is preferably selected from one or more of citric acid, anhydrous citric acid, tartaric acid, fumaric acid, sodium citrate, calcium hydrogenphosphate, calcium carbonate;
(5) a surfactant, which is preferably selected from one or more of polyethylene glycol, poloxamer, phospholipid, Tween 80, Span 40, propylene glycol monolaurate, sodium dodecyl sulfate;
(6) a lubricant, which is preferably selected from one or more of magnesium stearate, stearic acid, sodium stearyl fumarate, glyceryl behenate.

In some embodiments of the present invention, the oral pharmaceutical composition comprises:

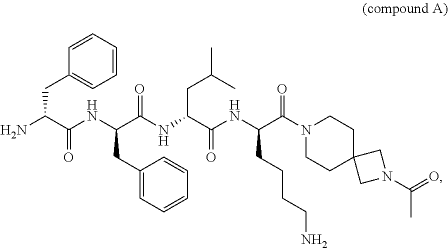 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from 4-[(4-chloro-2-hydroxy-benzoyl)amino]butyric acid or a pharmaceutically acceptable salt thereof.

In some embodiments of the present invention, the weight ratio of compound A to 4-[(4-chloro-2-hydroxy-benzoyl)amino]butyric acid or a pharmaceutically acceptable salt thereof is 1:20-1:200, preferably 1:40-1:80, further preferably 1:40.

In some embodiments of the present invention, the weight ratio of compound A to 4-[(4-chloro-2-hydroxy-benzoyl)amino]butyric acid or a pharmaceutically acceptable salt thereof is 1:20.

In some embodiments of the present invention, the weight ratio of compound A to 4-[(4-chloro-2-hydroxy-benzoyl)amino]butyric acid or a pharmaceutically acceptable salt thereof is 1:40.

In some embodiments of the present invention, the weight ratio of compound A to 4-[(4-chloro-2-hydroxy-benzoyl)amino]butyric acid or a pharmaceutically acceptable salt thereof is 1:80.

In some embodiments of the present invention, the weight ratio of compound A to 4-[(4-chloro-2-hydroxy-benzoyl)amino]butyric acid or a pharmaceutically acceptable salt thereof is 1:100.

In some embodiments of the present invention, the weight ratio of compound A to 4-[(4-chloro-2-hydroxy-benzoyl)amino]butyric acid or a pharmaceutically acceptable salt thereof is 1:200.

In some embodiments of the present invention, the weight ratio of compound A to 4-[(4-chloro-2-hydroxy-benzoyl)amino]butyric acid or a pharmaceutically acceptable salt thereof is 3:200.

In some embodiments of the present invention, the absorption enhancer is 4-[(4-chloro-2-hydroxy-benzoyl)amino]sodium butyrate.

In some embodiments of the present invention, the absorption enhancer is 4-[(4-chloro-2-hydroxy-benzoyl)amino]potassium butyrate.

In some embodiments of the present invention, the absorption enhancer is 4-[(4-chloro-2-hydroxy-benzoyl)amino]calcium butyrate.

In some embodiments of the present invention, the oral pharmaceutical composition comprises:

a) compound A:

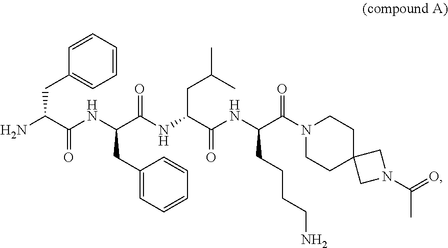 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from 4-[(4-chloro-2-hydroxy-benzoyl)amino]butyric acid or a pharmaceutically acceptable salt thereof;
c) one or more non-active ingredients selected from:
(1) a filler, which is preferably selected from one or more of microcrystalline cellulose, lactose, mannitol, anhydrous calcium hydrogenphosphate, pregelatinized starch, calcium hydrogenphosphate dihydrate;
(2) a binder, which is preferably selected from one or more of povidone, copovidone, a cellulose derivative, wherein the cellulose derivative is selected from one or more of hydroxypropylcellulose, hypromellose, methyl cellulose;
(3) a disintegrant, which is preferably selected from one or more of crospovidone, low substituted hydroxypropyl cellulose, croscarmellose sodium, sodium carboxymethyl starch;
(4) a pH regulator, which is preferably selected from one or more of citric acid, anhydrous citric acid, tartaric acid, fumaric acid, sodium citrate, calcium hydrogenphosphate, calcium carbonate;
(5) a surfactant, which is preferably selected from one or more of polyethylene glycol, poloxamer, phospholipid, Tween 80, Span 40, propylene glycol monolaurate, sodium dodecyl sulfate;
(6) a lubricant, which is preferably selected from one or more of magnesium stearate, stearic acid, sodium stearyl fumarate, glyceryl behenate.

In some embodiments of the present invention, the lubricant is magnesium stearate.

In some embodiments of the present invention, the filler is microcrystalline cellulose and/or anhydrous calcium hydrogenphosphate.

In some embodiments of the present invention, the binder is povidone.

In some embodiments of the present invention, the oral pharmaceutical composition comprises:

a) compound A:

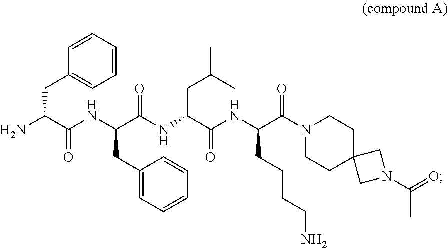 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) a lubricant.

In some embodiments of the present invention, the weight ratio of compound A:4-[(4-chloro-2-hydroxy-benzoyl)amino]butyric acid or a pharmaceutically acceptable salt thereof:the lubricant is 1:(20-200):(0.2-2).

In some embodiments of the present invention, the weight ratio of compound A:4-[(4-chloro-2-hydroxy-benzoyl)amino]butyric acid or a pharmaceutically acceptable salt thereof:the lubricant is 1:200:2.

In some embodiments of the present invention, the oral pharmaceutical composition comprises:

a) compound A:

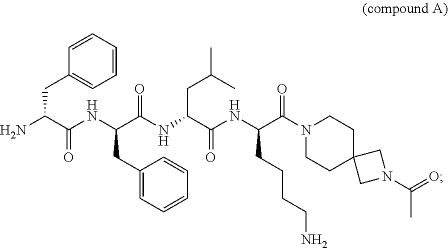 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) magnesium stearate.

In some embodiments of the present invention, the weight ratio of compound A:4-[(4-chloro-2-hydroxy-benzoyl)amino]butyric acid or a pharmaceutically acceptable salt thereof:magnesium stearate is 1:(20-200):(0.2-2).

In some embodiments of the present invention, the weight ratio of compound A:4-[(4-chloro-2-hydroxy-benzoyl)amino]butyric acid or a pharmaceutically acceptable salt thereof:magnesium stearate is 1:200:2.

In some embodiments of the present invention, the oral pharmaceutical composition comprises:

a) compound A:

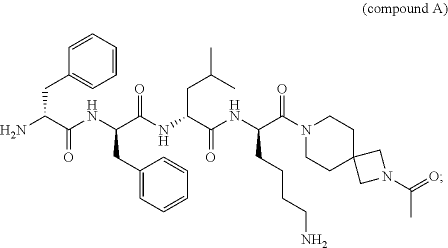 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) a filler;
d) a binder.

In some embodiments of the present invention, the weight ratio of compound A:4-[(4-chloro-2-hydroxy-benzoyl)amino]butyric acid or a pharmaceutically acceptable salt thereof:the filler:the binder is 1:(20-200):200:5.

In some embodiments of the present invention, the weight ratio of compound A:4-[(4-chloro-2-hydroxy-benzoyl)amino]butyric acid or a pharmaceutically acceptable salt thereof:the filler:the binder is 1:200:200:5.

In some embodiments of the present invention, the oral pharmaceutical composition comprises:

a) compound A:

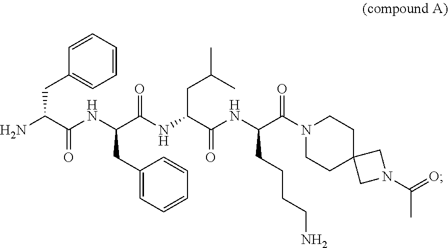 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) microcrystalline cellulose;
d) povidone.

In some embodiments of the present invention, the weight ratio of compound A:4-[(4-chloro-2-hydroxy-benzoyl)amino]butyric acid or a pharmaceutically acceptable salt thereof:microcrystalline cellulose:povidone is 1:(20-200):200:5.

In some embodiments of the present invention, the weight ratio of compound A:4-[(4-chloro-2-hydroxy-benzoyl)amino]butyric acid or a pharmaceutically acceptable salt thereof:microcrystalline cellulose:povidone is 1:200:200:5.

In some embodiments of the present invention, the oral pharmaceutical composition comprises:

a) compound A:

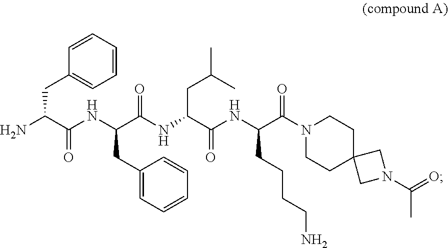 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) a filler;
d) a binder;
e) a lubricant.

In some embodiments of the present invention, the weight ratio of compound A:4-[(4-chloro-2-hydroxy-benzoyl)amino]butyric acid or a pharmaceutically acceptable salt thereof:the filler:the binder:the lubricant is 1:(20-200):200:5:(1-5).

In some embodiments of the present invention, the weight ratio of compound A:4-[(4-chloro-2-hydroxy-benzoyl)amino]butyric acid or a pharmaceutically acceptable salt thereof:the filler:the binder:the lubricant is 1:200:200:5:(4-5), for example 1:200:200:5:4.06.

In some embodiments of the present invention, the weight ratio of compound A:4-[(4-chloro-2-hydroxy-benzoyl)amino]butyric acid or a pharmaceutically acceptable salt thereof:the filler:the binder:the lubricant is 3:200:200:5:1.

In some embodiments of the present invention, the weight ratio of compound A:4-[(4-chloro-2-hydroxy-benzoyl)amino]butyric acid or a pharmaceutically acceptable salt thereof:the filler:the binder:the lubricant is 3:200:200:5:4.

In some embodiments of the present invention, the oral pharmaceutical composition comprises:

a) compound A:

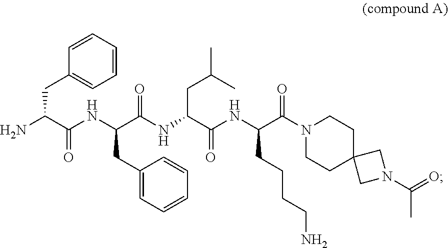 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) microcrystalline cellulose;
d) povidone;
e) magnesium stearate.

In some embodiments of the present invention, the weight ratio of compound A:4-[(4-chloro-2-hydroxy-benzoyl)amino]butyric acid or a pharmaceutically acceptable salt thereof:microcrystalline cellulose:povidone:magnesium stearate is (1-3):(20-200):200:5:(1-5).

In some embodiments of the present invention, the weight ratio of compound A:4-[(4-chloro-2-hydroxy-benzoyl)amino]butyric acid or a pharmaceutically acceptable salt thereof:microcrystalline cellulose:povidone:magnesium stearate is 1:200:200:5:(4-5), for example 1:200:200:5:4.06.

In some embodiments of the present invention, the weight ratio of compound A:4-[(4-chloro-2-hydroxy-benzoyl)amino]butyric acid or a pharmaceutically acceptable salt thereof:microcrystalline cellulose:povidone:magnesium stearate is 3:200:200:5:4.

In some embodiments of the present invention, the oral pharmaceutical composition comprises:

a) compound A:

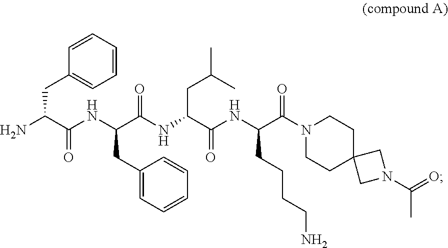 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) povidone;
d) anhydrous calcium hydrogenphosphate;
e) magnesium stearate.

In some embodiments of the present invention, the weight ratio of compound A:4-[(4-chloro-2-hydroxy-benzoyl)amino]butyric acid or a pharmaceutically acceptable salt thereof:povidone:anhydrous calcium hydrogenphosphate:magnesium stearate is (1-3):(20-200):5:200:(1-5).

In some embodiments of the present invention, the weight ratio of compound A:4-[(4-chloro-2-hydroxy-benzoyl)amino]butyric acid or a pharmaceutically acceptable salt thereof:povidone:anhydrous calcium hydrogenphosphate:magnesium stearate is 3:200:5:200:4.

In some embodiments of the present invention, the oral pharmaceutical composition comprises:

a) compound A:

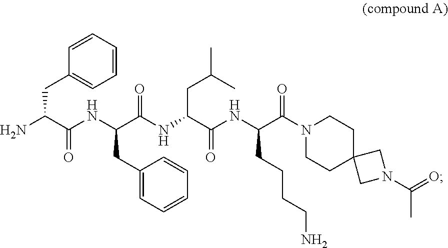 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) a surfactant;
d) a filler;
e) a lubricant.

In some embodiments of the present invention, the weight ratio of compound A:4-[(4-chloro-2-hydroxy-benzoyl)amino]butyric acid or a pharmaceutically acceptable salt thereof:the surfactant:the filler:the lubricant is (1-3):(20-200):(3-9):200:(1-5), for example 3:200:6:200:1.

In some embodiments of the present invention, the oral pharmaceutical composition comprises:

a) compound A:

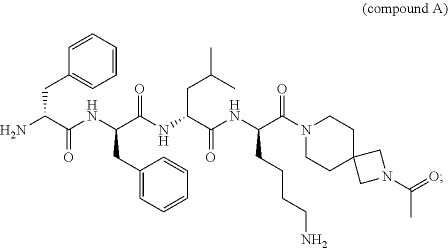 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) propylene glycol monolaurate and/or polyethylene glycol (for example polyethylene glycol 300);
d) anhydrous calcium hydrogenphosphate;
e) magnesium stearate.

In some embodiments of the present invention, the oral pharmaceutical composition comprises:

a) compound A:

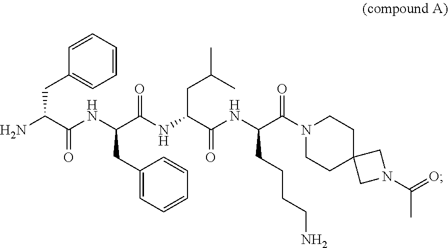 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

b) 4-[(4-chloro-2-hydroxy-benzoyl)amino]butyric acid or a pharmaceutically acceptable salt thereof;
c) propylene glycol monolaurate;
d) polyethylene glycol 300;
e) anhydrous calcium hydrogenphosphate;
f) magnesium stearate.

In some embodiments of the present invention, the weight ratio of compound A:4-[(4-chloro-2-hydroxy-benzoyl)amino]butyric acid or a pharmaceutically acceptable salt thereof:propylene glycol monolaurate:polyethylene glycol 300:anhydrous calcium hydrogenphosphate:magnesium stearate is (1-3):(20-200):(3-9):(3-9):200:(1-5), for example 3:200:3:3:200:1.

In some embodiments of the present invention, the oral pharmaceutical composition comprises:

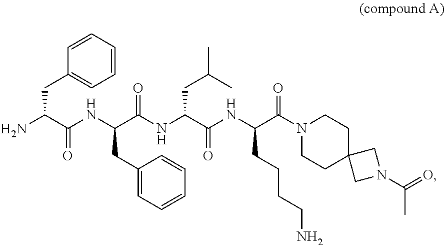 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from lauroyl-L-carnitine or a hydrochloride thereof.

In some embodiments of the present invention, the weight ratio of compound A to lauroyl-L-carnitine or a hydrochloride thereof is 1:10-1:150, for example 1:10, 1:15, 1:20, 1:30, 1:50, 1:75, 1:100 or 1:150.

In some embodiments of the present invention, the oral pharmaceutical composition comprises:

a) compound A:

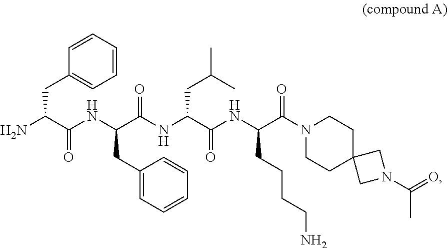 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from lauroyl-L-carnitine.

In some embodiments of the present invention, the weight ratio of compound A to lauroyl-L-carnitine is 1:10-1:50, preferably 1:10-1:20, further preferably 1:10.

In some embodiments of the present invention, the weight ratio of compound A to lauroyl-L-carnitine is 1:10.

In some embodiments of the present invention, the weight ratio of compound A to lauroyl-L-carnitine is 1:20.

In some embodiments of the present invention, the weight ratio of compound A to lauroyl-L-carnitine is 1:50.

In some embodiments of the present invention, the oral pharmaceutical composition comprises:

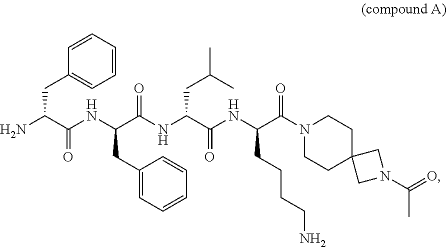 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from lauroyl-L-carnitine hydrochloride.

In some embodiments of the present invention, the weight ratio of compound A to lauroyl-L-carnitine hydrochloride is 1:15-1:150, preferably lauroyl-L-carnitine hydrochloride is used in an amount of 75-150 mg, further preferably lauroyl-L-carnitine hydrochloride is used in an amount of 75 mg.

In some embodiments of the present invention, the weight ratio of compound A to lauroyl-L-carnitine hydrochloride is 1:15.

In some embodiments of the present invention, the weight ratio of compound A to lauroyl-L-carnitine hydrochloride is 1:20.

In some embodiments of the present invention, the weight ratio of compound A to lauroyl-L-carnitine hydrochloride is 1:30.

In some embodiments of the present invention, the weight ratio of compound A to lauroyl-L-carnitine hydrochloride is 1:75.

In some embodiments of the present invention, the weight ratio of compound A to lauroyl-L-carnitine hydrochloride is 1:100.

In some embodiments of the present invention, the weight ratio of compound A to lauroyl-L-carnitine hydrochloride is 1:150.

In some embodiments of the present invention, when the absorption enhancer in the oral pharmaceutical composition is selected from lauroyl-L-carnitine or a hydrochloride thereof, the pharmaceutical composition does not comprise enteric coated excipients.

In some embodiments of the present invention, when the absorption enhancer in the oral pharmaceutical composition is selected from lauroyl-L-carnitine or a hydrochloride thereof, the pharmaceutical composition is not in the form of an enteric coated tablet or an enteric coated capsule.

In some embodiments of the present invention, when the absorption enhancer in the oral pharmaceutical composition is selected from lauroyl-L-carnitine or a hydrochloride thereof, the pharmaceutical composition is in the form of a gastric coated capsule, gastric coated tablet or gastric coated granule.

In some embodiments of the present invention, the oral pharmaceutical composition comprises:

a) compound A:

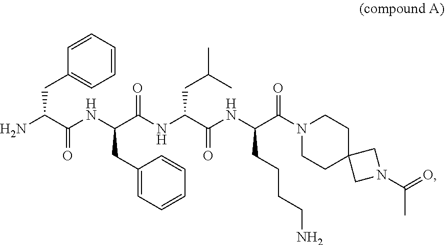 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from lauroyl-L-carnitine or a hydrochloride thereof;
c) one or more non-active ingredients selected from:
(1) a filler, which is preferably selected from one or more of microcrystalline cellulose, lactose, mannitol, anhydrous calcium hydrogenphosphate, pregelatinized starch, calcium hydrogenphosphate dihydrate;
(2) a binder, which is preferably selected from one or more of povidone, copovidone, a cellulose derivative, the cellulose derivative is selected from one or more of hydroxypropylcellulose, hypromellose, methyl cellulose;
(3) a disintegrant, which is preferably selected from one or more of crospovidone, low substituted hydroxypropyl cellulose, croscarmellose sodium, sodium carboxymethyl starch;
(4) a pH regulator, which is preferably selected from one or more of citric acid, anhydrous citric acid, tartaric acid, fumaric acid, sodium citrate, calcium hydrogenphosphate, calcium carbonate;
(5) a surfactant, which is preferably selected from one or more of polyethylene glycol, poloxamer, phospholipid, Tween 80, Span 40, propylene glycol monolaurate, sodium dodecyl sulfate;
(6) a lubricant, which is preferably selected from one or more of magnesium stearate, stearic acid, sodium stearyl fumarate, glyceryl behenate.

In some embodiments of the present invention, the oral pharmaceutical composition comprises:

a) compound A:

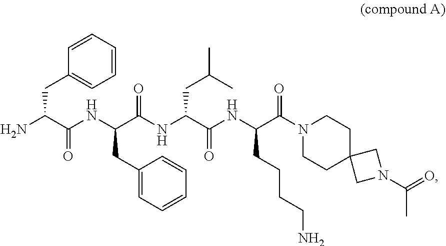 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) lauroyl-L-carnitine;
c) a pH regulator.
In some embodiments of the present invention, the weight ratio of compound A:lauroyl-L-carnitine:the pH regulator is 1:(10-50):24.

In some embodiments of the present invention, the oral pharmaceutical composition comprises:

a) compound A:

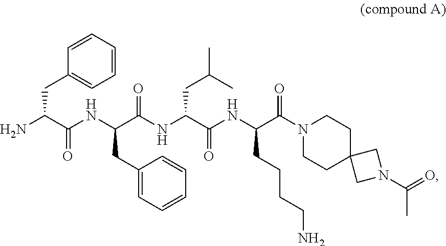 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) lauroyl-L-carnitine;
c) citric acid.

In some embodiments of the present invention, the weight ratio of compound A:lauroyl-L-carnitine:citric acid is 1:(10-50):24.

In some embodiments of the present invention, the weight ratio of compound A:lauroyl-L-carnitine:citric acid is 1:50:24.

In some embodiments of the present invention, the oral pharmaceutical composition comprises:

a) compound A:

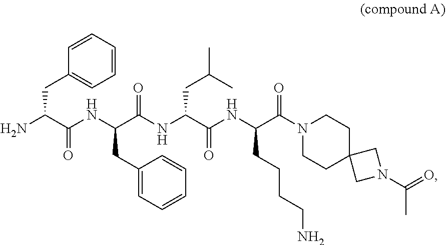 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from sodium caprate.
In some embodiments of the present invention, the weight ratio of compound A to sodium caprate is 1:50-1:200, preferably 1:100-1:200, most preferably 1:100.

In some embodiments of the present invention, the weight ratio of compound A to sodium caprate is 1:50.

In some embodiments of the present invention, the weight ratio of compound A to sodium caprate is 1:100.

In some embodiments of the present invention, the weight ratio of compound A to sodium caprate is 1:200.

In some embodiments of the present invention, the oral pharmaceutical composition comprises:

a) compound A:

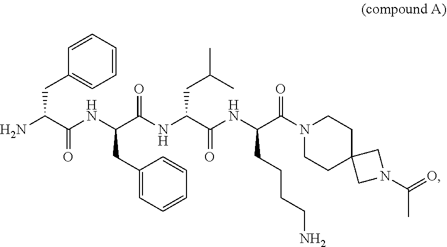 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from sodium caprate;
c) one or more non-active ingredients selected from:
(1) a filler, which is preferably selected from one or more of microcrystalline cellulose, lactose, mannitol, anhydrous calcium hydrogenphosphate, pregelatinized starch, calcium hydrogenphosphate dihydrate;
(2) a binder, which is preferably selected from one or more of povidone, copovidone, a cellulose derivative, wherein the cellulose derivative is selected from one or more of hydroxypropylcellulose, hypromellose, methyl cellulose;
(3) a disintegrant, which is preferably selected from one or more of crospovidone, low substituted hydroxypropyl cellulose, croscarmellose sodium, sodium carboxymethyl starch;
(4) a glyceride, which is preferably selected from one or more of monocaprylin, tricaprylin.
(5) a surfactant, which is preferably selected from one or more of caprylocaproyl macrogolglyceride, polyethylene glycol, poloxamer, phospholipid, Tween 80, Span 40, propylene glycol monolaurate, sodium dodecyl sulfate;
(6) a lubricant, which is preferably selected from one or more of magnesium stearate, stearic acid, sodium stearyl fumarate, glyceryl behenate.

In some embodiments of the present invention, the binder is povidone.

In some embodiments of the present invention, the oral pharmaceutical composition comprises:

a) compound A:

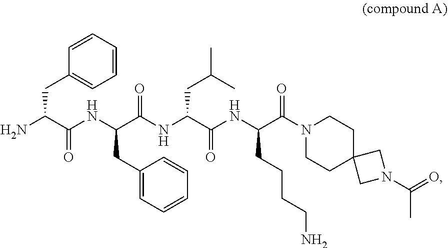 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) sodium caprate;
c) povidone.

In some embodiments of the present invention, the weight ratio of compound A:sodium caprate:povidone is 1:(50-200):50, for example 1:50:50, 1:100:50 or 1:200:50.

In some embodiments of the present invention, the oral pharmaceutical composition comprises:

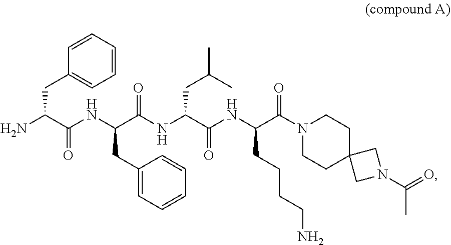 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from capric acid;
c) one or more non-active ingredients selected from:
(1) a filler, preferably the filler is selected from one or more of microcrystalline cellulose, lactose, mannitol, anhydrous calcium hydrogenphosphate, pregelatinized starch, calcium hydrogenphosphate dihydrate;
(2) a binder, which is preferably selected from one or more of povidone, copovidone, a cellulose derivative, wherein the cellulose derivative is selected from one or more of hydroxypropylcellulose, hypromellose, methyl cellulose;
(3) a disintegrant, which is preferably selected from one or more of crospovidone, low substituted hydroxypropyl cellulose, croscarmellose sodium, sodium carboxymethyl starch;
(4) a glyceride, which is preferably selected from one or more of monocaprylin, tricaprylin. (5) a surfactant, preferably the surfactant is selected from one or more of caprylocaproyl macrogolglyceride, polyethylene glycol, poloxamer, phospholipid, Tween 80, Span 40, propylene glycol monolaurate, sodium dodecyl sulfate;
(6) a lubricant, which is preferably selected from one or more of magnesium stearate, stearic acid, sodium stearyl fumarate, glyceryl behenate.

In some embodiments of the present invention, the oral pharmaceutical composition comprises:

a) compound A:

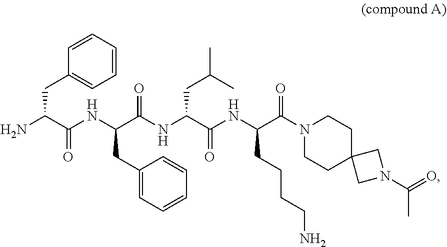 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from caprylocaproyl macrogolglyceride.

In some embodiments of the present invention, the weight ratio of compound A to caprylocaproyl macrogolglyceride is 1:600-1:3600, preferably 1:2400-1:3600, most preferably 1:2400.

In some embodiments of the present invention, the weight ratio of compound A to caprylocaproyl macrogolglyceride is 1:600.

In some embodiments of the present invention, the weight ratio of compound A to caprylocaproyl macrogolglyceride is 1:1200.

In some embodiments of the present invention, the weight ratio of compound A to caprylocaproyl macrogolglyceride is 1:1800.

In some embodiments of the present invention, the weight ratio of compound A to caprylocaproyl macrogolglyceride is 1:2400.

In some embodiments of the present invention, the weight ratio of compound A to caprylocaproyl macrogolglyceride is 1:3600.

In some embodiments of the present invention, the oral pharmaceutical composition comprises:

a) compound A:

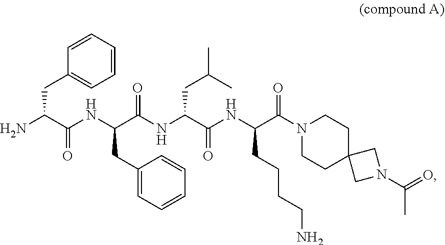 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from caprylocaproyl macrogolglyceride;
c) one or more non-active ingredients selected from:
(1) a hydrophobic medium, which is preferably selected from one or more of coconut oil, castor oil, olive oil;
(2) a surfactant, which is preferably selected from one or more of polyethylene glycol, poloxamer, phospholipid, Tween 80, Span 40, propylene glycol monolaurate, sodium dodecyl sulfate;
(3) a glyceride, which is preferably selected from one or more of monocaprylin, tricaprylin.

In some embodiments of the present invention, the oral pharmaceutical composition comprises:

a) compound A:

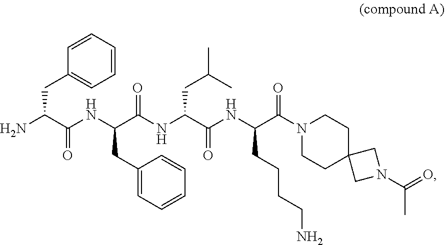 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) an absorption enhancer selected from caprylocaproyl macrogolglyceride;
c) one or more non-active ingredients selected from:
(1) a hydrophobic medium, which is preferably selected from one or more of coconut oil, castor oil, olive oil;
(2) a surfactant, which is preferably selected from one or more of polyethylene glycol, poloxamer, phospholipid, Tween 80, Span 40, propylene glycol monolaurate, sodium dodecyl sulfate;
(3) a glyceride other than caprylocaproyl macrogolglyceride, preferably the glyceride is selected from one or more of monocaprylin, tricaprylin.

In some embodiments of the present invention, the oral pharmaceutical composition comprises:

a) compound A:

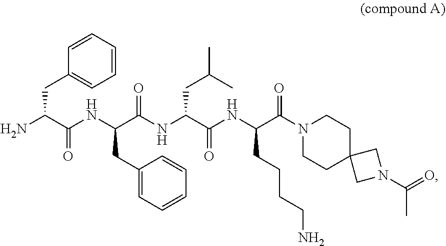 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]

and
b) caprylocaproyl macrogolglyceride;
c) capric acid.

In some embodiments of the present invention, the weight ratio of compound A:caprylocaproyl macrogolglyceride:capric acid is 1:(3000-6000):(50-200), for example 1:3000:(50-100) or 1:(4000-6000):200, for example 1:3000:50, 1:3000:75, 1:3000:100, 1:4000:200 or 1:6000:200.

In some embodiments of the present invention, the oral pharmaceutical composition is in the form of a solution, a suspension, a granule, a powder, a capsule, a tablet or other oral dosage forms.

In some embodiments of the present invention, the oral pharmaceutical composition is in the form of a gastric coated capsule, a gastric coated tablet or a gastric coated granule.

In some embodiments of the present invention, compound (A) is present in the oral pharmaceutical composition at a therapeutically effective amount or unit dose. In some embodiments of the present invention, compound (A) is present in the oral pharmaceutical composition at a does of less than 1 mg, 1-3 mg, 3-5 mg, 5-10 mg, 10-25 mg, 25-32 mg, 32-100 mg, or more than 100 mg.

In some embodiments of the present invention, the above-mentioned oral pharmaceutical composition is prepared as follows: compound A, the absorption enhancer and other non-active ingredients are mixed directly, and then filled into capsules or compressed into tablets.

In some embodiments of the present invention, the above-mentioned oral pharmaceutical composition is prepared as follows: compound A, the absorption enhancer and other non-active ingredients are wet granulated and then filled into capsules or compressed into tablets.

In some embodiments of the present invention, the above-mentioned oral pharmaceutical composition is prepared as follows: compound A, the absorption enhancer and the hydrophilic non-active ingredients are wet granulated, then dispersed in a hydrophobic medium, and filled into capsules.

In the present invention, the oral pharmaceutical composition can also be prepared as a solution, a suspension, a granule, a powder or other oral dosage forms using a conventional preparation process.

In the present invention, the capsule may be a hard capsule or a soft capsule.

The present invention also provides the use of the oral pharmaceutical composition in preparing a drug for treating diseases or conditions related to the κ-opioid receptor.

The present invention also provides a method for treating diseases or conditions related to the κ-opioid receptor in a subject, comprising administering the oral pharmaceutical composition of any one of the foregoing embodiments to the subject.

In some embodiments of the present invention, the diseases or conditions related to the κ-opioid receptor are selected from pain, inflammation, itching, edema, hyponatremia, hypokalemia, intestinal obstruction, cough and glaucoma.

In some embodiments of the present invention, the pain is selected from neuropathic pain, somatic pain, visceral pain and skin pain.

In some embodiments of the present invention, the diseases or conditions are selected from arthritis pain, kidney stone pain, hysterotrismus, dysmenorrhea, endometriosis, dyspepsia, pain after surgery, pain after medical treatment, ocular pain, otitis pain, breakthrough cancer pain, and pain associated with GI disorders (gastrointestinal disorders).

In one embodiment of the present invention, the oral pharmaceutical composition can be used for treating acute and chronic pain and itching.

In certain embodiments, the oral pharmaceutical composition is administered to the subject 1, 2, or 3 times daily.

In certain embodiments, the subject is a mammal, such as a bovine, an equine, a porcine, a canine, a feline, a rodent, a primate animal, wherein, particularly preferred subjects are humans.

### DETAILED DESCRIPTION OF EMBODIMENTS

Unless stated to the contrary, the terms used in the description and claims have the following meanings.

(1) SNAC: sodium N-[8-(2-hydroxybenzoyl)amino]octanoate.
(2) 4-CNAB: sodium 4-[(4-chloro-2-hydroxy-benzoyl)amino]butyrate.

The implementation process and beneficial effects of the present invention are described in detail below through specific examples, which are intended to help readers better understand the essence and characteristics of the present invention, and are not intended to limit the scope of implementation of the present invention.

The raw materials/reagents used in the examples are commercially available or self-prepared, and the formulations in the examples are all at single preparation dosage.

Unless otherwise specified, “0 #capsule” in the following examples refers to the shell that can be used to prepare gastric coated hard capsules with the specification of 0 #.

Compound A was prepared with reference to the method disclosed in WO 2019/015644.

### Example 1

3 mg of compound A was dissolved in physiological saline to prepare 0.06 mg/ml physiological saline solution.

### Example 2

5 mg of compound A and 150 mg of SNAC were mixed uniformly and then filled into 0 #capsules.

### Example 3

5 mg of compound A and 100 mg of SNAC were mixed uniformly and then filled into 0 #capsules.

### Example 4

5 mg of compound A and 200 mg of SNAC were mixed uniformly and then filled into 0 #capsules.

### Example 5

5 mg of compound A and 400 mg of SNAC were mixed uniformly and then filled into 0 #capsules.

### Example 6

5 mg of compound A, 50 mg of lauroyl-L-carnitine and 120 mg of citric acid were mixed uniformly and then filled into 0 #capsules.

### Example 7

5 mg of compound A and 100 mg of 4-CNAB were mixed uniformly and then filled into 0 #capsules.

### Example 8

5 mg of compound A and 200 mg of 4-CNAB were mixed uniformly and then filled into 0 #capsules.

### Example 9

5 mg of compound A and 400 mg of 4-CNAB were mixed uniformly and then filled into 0 #capsules.

### Example 10

1 mg of compound A and 200 mg of 4-CNAB were mixed uniformly and then filled into 0 #capsules.

### Example 11

1 mg of compound A, 200 mg of 4-CNAB and 2 mg of magnesium stearate were mixed uniformly and then compressed into tablets.

### Example 12

1 mg of compound A, 200 mg of 4-CNAB, 200 mg of microcrystalline cellulose, and 5 mg of povidone were mixed uniformly and then filled into 0 #capsules.

### Example 13

1 mg of compound A, 200 mg of 4-CNAB, 200 mg of microcrystalline cellulose, 5 mg of povidone, and 4.06 mg of magnesium stearate were mixed uniformly and then compressed into tablets.

### Example 14

1 mg of compound A, 200 mg of 4-CNAB, 200 mg of microcrystalline cellulose, and 5 mg of povidone were weighed; compound A and 4-CNAB were dissolved in water, subjected to rotary evaporation, dried, ground and pulverized; and same were mixed with the other non-active ingredients uniformly and then filled into 0 #capsules.

### Example 15

1 mg of compound A, 200 mg of 4-CNAB, 200 mg of microcrystalline cellulose, 5 mg of povidone, and 4.06 mg of magnesium stearate were weighed; compound A and 4-CNAB were dissolved in water, subjected to rotary evaporation, dried, ground and pulverized; and same were mixed with the other non-active ingredients and then compressed into tablets.

### Example 16

2 mg of compound A and 200 mg of 4-CNAB were mixed uniformly and then filled into 0 #capsules.

### Example 17

3 mg of compound A and 200 mg of 4-CNAB were mixed uniformly and then filled into 0 #capsules.

### Example 18

10 mg of compound A and 200 mg of 4-CNAB were mixed uniformly and then filled into 0 #capsules.

### Example 19

3 mg of compound A, 200 mg of 4-CNAB, 5 mg of povidone, 200 mg of microcrystalline cellulose, and 4 mg of magnesium stearate were mixed uniformly and then filled into 0 #capsules.

### Example 20

3 mg of compound A, 200 mg of 4-CNAB, 5 mg of povidone, 200 mg of anhydrous calcium hydrogenphosphate, and 4 mg of magnesium stearate were mixed uniformly and then filled into 0 #capsules.

### Example 21

\textcircled{1} 3 mg of compound A and 200 mg of 4-CNAB were mixed uniformly.

\textcircled{2} 3 mg of propylene glycol monolaurate and 3 mg of polyethylene glycol 300 were absorbed by and dispersed with 200 mg of anhydrous calcium hydrogenphosphate.

\textcircled{3} The samples from the above two steps were mixed uniformly.

\textcircled{4} 1 mg of magnesium stearate was added, and the mixture was mixed uniformly and then filled into 0 #capsules.

### Example 22

100 mg of compound A, 5 g of povidone K12 and 5 g of sodium caprate were completely dissolved in 100 g of purified water, then freeze-dried and passed through a 120-mesh sieve and then filled into 0 #capsules.

### Example 23

100 mg of compound A, 5 g of povidone K12 and 10 g of sodium caprate were completely dissolved in 100 g of purified water, then freeze-dried and passed through a 120-mesh sieve and then filled into 0 #capsules.

### Example 24

25 mg of compound A, 1.25 g of povidone K12 and 5 g of sodium caprate were completely dissolved in 100 g of purified water, then freeze-dried and passed through a 120-mesh sieve and then filled into 00 #capsules.

### Example 25

1 mg of compound A and 0.6 g of caprylocaproyl macrogolglyceride were completely dissolved in purified water to obtain a 10 ml solution.

### Example 26

1 mg of compound A and 1.2 g of caprylocaproyl macrogolglyceride were completely dissolved in purified water to obtain a 10 ml solution.

### Example 27

1 mg of compound A and 1.8 g of caprylocaproyl macrogolglyceride were completely dissolved in purified water to obtain a 10 ml solution.

### Example 28

1 mg of compound A and 2.4 g of caprylocaproyl macrogolglyceride were completely dissolved in purified water to obtain a 10 ml solution.

### Example 29

1 mg of compound A and 3.6 g of caprylocaproyl macrogolglyceride were completely dissolved in purified water to obtain a 10 ml solution.

### Example 30

1 mg of compound A and 0.6 g of caprylocaproyl macrogolglyceride were mixed and suspended uniformly by ultrasonic dispersion, and then filled into 0 #capsules.

### Example 31

1 mg of compound A and 1.8 g of caprylocaproyl macrogolglyceride were mixed and suspended uniformly by ultrasonic dispersion, and then filled into 0 #capsules.

### Example 32

5 mg of compound A and 3.0 g of caprylocaproyl macrogolglyceride were mixed and suspended uniformly by ultrasonic dispersion, and then filled into 0 #capsules.

### Example 33

1 mg of compound A, 3 g of caprylocaproyl macrogolglyceride and 50 mg of sodium caprate were completely dissolved in purified water to obtain a 10 ml solution.

### Example 34

1 mg of compound A, 3 g of caprylocaproyl macrogolglyceride and 75 mg of sodium caprate were completely dissolved in purified water to obtain a 10 ml solution.

### Example 35

1 mg of compound A, 3 g of caprylocaproyl macrogolglyceride and 100 mg of sodium caprate were completely dissolved in purified water to obtain a 10 ml solution.

### Example 36

1 mg of compound A, 4 g of caprylocaproyl macrogolglyceride and 200 mg of mgcapric acid were completely dissolved in purified water to obtain a 10 ml solution.

### Example 37

1 mg of compound A, 6 g of caprylocaproyl macrogolglyceride and 200 mg of mgcapric acid were completely dissolved in purified water to obtain a 10 ml solution.

### Example 38

5 mg of compound A and 50 mg of lauroyl-L-carnitine were filled into 0 #capsules.

### Example 39

5 mg of compound A and 100 mg of lauroyl-L-carnitine were filled into 0 #capsules.

### Example 40

5 mg of compound A and 250 mg of lauroyl-L-carnitine were filled into 0 #capsules.

### Example 41

5 mg of compound A and 100 mg of lauroyl-L-carnitine were filled into 0 #enteric coated capsules.

### Example 42

5 mg of compound A and 75 mg of lauroyl-L-carnitine hydrochloride were filled into 0 #capsules.

### Example 43

5 mg of compound A and 100 mg of lauroyl-L-carnitine hydrochloride were filled into 0 #capsules.

### Example 44

5 mg of compound A and 150 mg of lauroyl-L-carnitine hydrochloride were filled into 0 #capsules.

### Example 45

1 mg of compound A and 75 mg of lauroyl-L-carnitine hydrochloride were filled into 0 #capsules.

### Example 46

1 mg of compound A and 100 mg of lauroyl-L-carnitine hydrochloride were filled into 0 #capsules.

### Example 47

1 mg of compound A and 150 mg of lauroyl-L-carnitine hydrochloride were filled into 0 #capsules.

### Example 48

1 mg of compound A and 200 mg of sodium caproate were filled into 0 #capsules.

### Example 49

1 mg of compound A and 100 mg of sodium caprylate were filled into 0 #capsules.

### Example 50

1 mg of compound A and 200 mg of sodium laurate were filled into 0 #capsules.

### Example 51

1 mg of compound A and 200 mg of sodium myristate were filled into 0 #capsules.

### Example 52

1 mg of compound A and 200 mg of sodium palmitate were filled into 0 #capsules.

### Test Example 1

The physiological saline solution of compound A obtained in Example 1 was orally administered at a single dose to male Beagle dogs (3 for each preparation), venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

After a single oral administration of the capsules obtained in Example 2 to male Beagle dogs (3 for each preparation) with 5 tablets of the samples for each dog, venous blood was taken at 0, 0.083, 0.25, 0.5, 1, 2, 4, 6, 8 and 24 h, and the absolute bioavailability (F %) (relative to intravenous dose of compound A) was calculated from the area under the curve obtained from the points where the plasma concentration of compound A was taken as a function of time.

TABLE 1
Effect of SNAC on absorption of compound A

| Formula | Cₘₐₓ (ng/ml) | F % |
|---|---|---|
| Example 1 | 2.60 ± 0.49 | 1.81 ± 0.01 |
| Example 2 | 125 ± 47 | 3.46 ± 1.62 |

### Results and Discussion:

By means of adding 150 mg of SNAC to the formulations, the bioavailability is significantly improved, and the absolute bioavailability is up to 3.46%.

### Test Example 2

After a single oral administration of the capsules obtained in Examples 3-5 to male Beagle dogs (3 for each preparation) with 1 tablet of the samples for each dog, venous blood was taken at 0, 0.083, 0.25, 0.5, 0.75, 1, 2, 4, 8, 10, 12 and 24 h, and the absolute bioavailability (F %) (relative to intravenous dose of compound A) was calculated from the area under the curve obtained from the points where the plasma concentration of compound A was taken as a function of time.

TABLE 2
Effect of dosage of SNAC on absorption of compound A

| Formula | Cₘₐₓ (ng/ml) | F % |
|---|---|---|
| Example 1 | 2.60 ± 0.49 | 1.81 ± 0.01 |
| Example 3 | 6.88 ± 5.8 | 2.18 ± 1.3 |
| Example 4 | 109 ± 56 | 9.82 ± 3.8 |
| Example 5 | 41.2 ± 22 | 5.60 ± 3.3 |

### Results and Discussion:

In the case of the weight ratio of compound A to SNAC being in the range of 1:20-1:80, the bioavailability of compound A can be significantly improved, and the absolute bioavailability is better when the weight ratio thereof is 1:40.

### Test Example 3

After a single oral administration of the capsules obtained in Example 6 to male Beagle dogs (3 for each preparation) with 5 tablets of the samples for each dog, venous blood was taken at 0, 0.25, 0.5, 1, 2, 4, 6, 8, 12, 24, 28, 48 and 72 h, and the absolute bioavailability (F %) (relative to intravenous dose of compound A) was calculated from the area under the curve obtained from the points where the plasma concentration of compound A was taken as a function of time.

TABLE 3
Effect of formula on absorption of compound A

| Formula | Cₘₐₓ (ng/ml) | F % |
|---|---|---|
| Example 1 | 2.60 ± 0.49 | 1.81 ± 0.01 |
| Example 6 | 451 ± 384 | 6.62 ± 5.3 |

### Results and Discussion:

Adding 50 mg of lauroyl-L-carnitine and 120 mg of citric acid to the formulations provides a good absorption promoting effect, and the absolute bioavailability of compound A is up to 6.62%.

### Test Example 4

After a single oral administration of the capsules obtained in Examples 7-9 to male Beagle dogs (3 for each preparation) with 1 tablet of the samples for each dog, venous blood was taken at 0, 0.083, 0.25, 0.5, 0.75, 1, 2, 4, 8, 10, 12 and 24 h, and the absolute bioavailability (F %) (relative to intravenous dose of compound A) was calculated from the area under the curve obtained from the points where the plasma concentration of compound A was taken as a function of time.

TABLE 4
Effect of dosage of 4-CNAB on absorption of compound A

| Formula | Cₘₐₓ (ng/ml) | F % |
|---|---|---|
| Example 1 | 2.60 ± 0.49 | 1.81 ± 0.01 |
| Example 7 | 61.7 ± 48 | 4.83 ± 2.7 |
| Example 8 | 166 ± 54 | 10.5 ± 1.6 |
| Example 9 | 80.7 ± 70 | 6.63 ± 4.5 |

### Results and Discussion:

In the case of the weight ratio of compound A to 4-CNAB being in the range of 1:20-1:80, the bioavailability of compound A can be significantly improved, and the absolute bioavailability is better when the weight ratio thereof is 1:40.

### Test Example 5

After a single oral administration of the preparation samples prepared in Examples 10-15 by different processes to male Beagle dogs (3 for each preparation) with 1 tablet of the samples for each dog, venous blood was taken at 0, 0.083, 0.25, 0.5, 1, 2, 4, 6, 8, 12, 24, 28 and 48 h, and the absolute bioavailability (F %) (relative to intravenous dose of compound A) was calculated from the area under the curve obtained from the points where the plasma concentration of compound A was taken as a function of time.

TABLE 5
Effect of different preparation processes on absorption of compound A

| Formula | Preparation method | Cₘₐₓ (ng/ml) | F % |
|---|---|---|---|
| Example 1 | Dissolving in physiological saline | 2.60 ± 0.49 | 1.81 ± 0.01 |
| Example 10 | Mixing uniformly and then filling into capsules | 15.8 ± 11 | 6.14 ± 4.1 |
| Example 11 | Mixing uniformly and then compressing into tablets | 14.5 ± 15 | 5.57 ± 5.5 |
| Example 12 | Mixing uniformly and then filling into capsules | 24.1 ± 19 | 8.57 ± 7.1 |
| Example 13 | Mixing uniformly and then compressing into tablets | 22.0 ± 7.4 | 7.91 ± 2.9 |
| Example 14 | Compound A and 4-CNAB were dissolved in water, | 18.4 ± 3.2 | 5.70 ± 0.46 |
| | subjected to rotary evaporation, dried, ground and | | |
| | pulverized, and same were mixed with other | | |
| | excipients and then filled into capsules | | |
| Example 15 | Compound A and 4-CNAB were dissolved in water, | 14.2 ± 3.6 | 6.28 ± 1.9 |
| | subjected to rotary evaporation, dried, ground and | | |
| | pulverized, and same were mixed with other | | |
| | excipients and then compressed into tablets | | |

### Results and Discussion:

By means of adding 4-CNAB to the formulations, the absolute bioavailability of samples at different dosage forms and prepared by different preparation processes is increased significantly.

### Test Example 6

After a single oral administration of the preparation samples obtained in Examples 22-24 to male Beagle dogs (3 for each preparation) with 1 tablet of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 6
Effect of dosage of sodium caprate on absorption of compound A

| Formula | Cₘₐₓ (ng/ml) | F % |
|---|---|---|
| Example 1 | 2.60 ± 0.49 | 1.81 ± 0.01 |
| Example 22 | 1.53 ± 0.19 | 1.36 ± 0.15 |
| Example 23 | 9.10 ± 3.7 | 5.59 ± 2.0 |
| Example 24 | 15.9 ± 14 | 5.73 ± 3.3 |

### Results and Discussion:

In the formulations, the bioavailability of compound A is improved by sodium caprate in a dose-response manner. When the weight ratio of compound A to sodium caprate is 1:50, the bioavailability does not change significantly, while the weight ratio of compound A to sodium caprate is 1:100 and 1:200, the bioavailability is increased significantly.

### Test Example 7

After a single oral administration of the preparation samples obtained in Examples 25-29 to male Beagle dogs (3 for each preparation) with 10 ml of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 7
Effect of dosage of caprylocaproyl macrogolglyceride
on absorption of compound A

| Formula | Cₘₐₓ (ng/ml) | F % |
|---|---|---|
| Example 1 | 2.60 ± 0.49 | 1.81 ± 0.01 |
| Example 25 | 20.0 ± 3.4 | 6.97 ± 2.7 |
| Example 26 | 16.3 ± 4.0 | 6.53 ± 1.4 |
| Example 27 | 17.4 ± 7.5 | 6.75 ± 2.6 |
| Example 28 | 20.6 ± 4.9 | 9.38 ± 1.6 |
| Example 29 | 21.9 ± 18 | 7.73 ± 4.6 |

### Results and Discussion:

In the formulations, the bioavailability is significantly improved when the weight ratio of compound A to caprylocaproyl macrogolglyceride is 1:600-1:3600, and the absolute bioavailability is better when the weight ratio is 1:2400.

### Test Example 8

After a single oral administration of the preparation samples obtained in Examples 25, 27 and 30-32 to male Beagle dogs (3 for each preparation), venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 8
Effect of dosage forms and specification
on absorption of compound A

| Formula | Cₘₐₓ (ng/ml) | F % |
|---|---|---|
| Example 1 | 2.60 ± 0.49 | 1.81 ± 0.01 |
| Example 25 | 20.0 ± 3.4 | 6.97 ± 2.7 |
| Example 30 | 28.0 ± 11 | 8.63 ± 2.5 |
| Example 27 | 17.4 ± 7.5 | 6.75 ± 2.6 |
| Example 31 | 17.8 ± 4.7 | 6.47 ± 1.8 |
| Example 32 | 65.1 ± 40 | 6.97 ± 4.7 |

### Results and Discussion:

Both solutions and capsules obtained by adding caprylocaproyl macrogolglyceride to the formulations can significantly improve the bioavailability of compound A, and the improvement levels of the bioavailability of compound A are similar at the specification of 1 mg and 5 mg.

### Test Example 9

After a single oral administration of the preparation samples obtained in Examples 33-35 to male Beagle dogs (3 for each preparation) with 10 ml of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 9
Effect of dosage of caprylocaproyl macrogolglyceride
and sodium caprate on absorption of compound A

| Formula | Cₘₐₓ (ng/ml) | F % |
|---|---|---|
| Example 1 | 2.60 ± 0.49 | 1.81 ± 0.01 |
| Example 33 | 17.9 ± 12 | 7.52 ± 5.1 |
| Example 34 | 16.1 ± 7.3 | 6.95 ± 2.6 |
| Example 35 | 23.2 ± 4.5 | 12.2 ± 2.9 |

### Results and Discussion:

In the formulations, the bioavailability of compound A is significantly improved when the weight ratio of compound A:caprylocaproyl macrogolglyceride:sodium caprate is 1:3000:50-100, and the bioavailability is better when the weight ratio is 1:3000:100.

### Test Example 10

After a single oral administration of the preparation samples obtained in Examples 36-37 to male Beagle dogs (3 for each preparation) with 10 ml of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 10
Effect of dosage of caprylocaproyl macrogolglyceride
and capric acid on absorption of compound A

| Formula | Cₘₐₓ (ng/ml) | F % |
|---|---|---|
| Example 1 | 2.60 ± 0.49 | 1.81 ± 0.01 |
| Example 36 | 15.0 ± 5.8 | 6.02 ± 1.7 |
| Example 37 | 15.1 ± 1.7 | 7.86 ± 1.6 |

In the formulations, the bioavailability of compound A is significantly improved when the weight ratio of compound A:caprylocaproyl macrogolglyceride:capric acid is 1:(4000-6000):200, and the bioavailability is better when the weight ratio is 1:6000:200.

### Test Example 11

After a single oral administration of the preparation samples obtained in Examples 43-45 to male Beagle dogs (3 for each preparation) with 1 tablet of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 11
Effect of dosage of lauroyl-L-carnitine
on absorption of compound A

| Formula | Cₘₐₓ (ng/ml) | F % |
|---|---|---|
| Example 1 | 2.60 ± 0.49 | 1.81 ± 0.01 |
| Example 38 | 56.6 ± 82 | 5.33 ± 5.9 |
| Example 39 | 232 ± 124 | 16.7 ± 5.2 |
| Example 40 | 105 ± 103 | 8.92 ± 8.0 |
| Example 41 | 62.8 ± 13 | 4.05 ± 0.48 |

### Results and Discussion:

In the formulations, the bioavailability of compound A is significantly improved when the weight ratio of compound A to lauroyl-L-carnitine is 1:10-1:50, and the bioavailability is better when the weight ratio is 1:20.

When the weight ratio of compound A to lauroyl-L-carnitine is 1:20, the bioavailability of the gastric coated capsule preparation is 4 times that of the enteric coated capsule preparation.

### Test Example 12

After a single oral administration of the preparation samples obtained in Examples 42-47 to male Beagle dogs (3 for each preparation) 1 tablet of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 12
Effect of dosage of lauroyl-L-camitine hydrochloride
on absorption of compound A

| Formula | Cₘₐₓ (ng/ml) | F % |
|---|---|---|
| Example 1 | 2.60 ± 0.49 | 1.81 ± 0.01 |
| Example 42 | 117 ± 49 | 11.8 ± 6.1 |
| Example 43 | 90.1 ± 107 | 8.32 ± 9.3 |
| Example 44 | 125 ± 33 | 8.30 ± 1.3 |
| Example 45 | 25.4 ± 25 | 12.4 ± 8.9 |
| Example 46 | 16.5 ± 8.3 | 9.17 ± 5.3 |
| Example 47 | 24.1 ± 20 | 6.04 ± 4.1 |

### Results and Discussion:

The bioavailability of compound A is significantly improved when the weight ratio of compound A to lauroyl-L-carnitine hydrochloride is 1:15-1:150, and the bioavailability is better when the weight ratio is 1:15 and 1:75.

### Test Example 13

After a single oral administration of the preparation samples obtained in Examples 24 and 48-52 to male Beagle dogs (3 for each preparation) 1 tablet of the samples for each dog, venous blood was taken at 0, 0.25, 0.5, 1, 2, 4, 6, 8, 10, 12 and 24 h, and the absolute bioavailability (F %) (relative to intravenous dose of compound A) was calculated from the area under the curve obtained from the points where the plasma concentration of compound A was taken as a function of time.

TABLE 13
Effect of types of fatty acid salts on absorption of compound A

| Formula | Cₘₐₓ (ng/ml) | F % |
|---|---|---|
| Example 1 | 2.60 ± 0.49 | 1.81 ± 0.01 |
| Example 24 | 15.9 ± 14 | 5.73 ± 3.3 |
| Example 48 | 1.80 ± 1.1 | 0.792 ± 0.31 |
| Example 49 | 13.7 ± 4.11 | 1.58 ± 1.11 |
| Example 50 | 3.54 ± 1.1 | 1.53 ± 0.31 |
| Example 51 | 0.837 ± 0.48 | 1.22 ± 0.38 |
| Example 52 | 1.48 ± 1.0 | 1.36 ± 0.22 |

### Results and Discussion:

Fatty acid salts with different aliphatic chain lengths have different effects on bioavailability. Among the fatty acid salts with aliphatic chain lengths of C₆-C₁₆, only the fatty acid salts with aliphatic chain length of C₁₀ can significantly increase the bioavailability of compound A.

1. An oral pharmaceutical composition, comprising:
a) compound A: 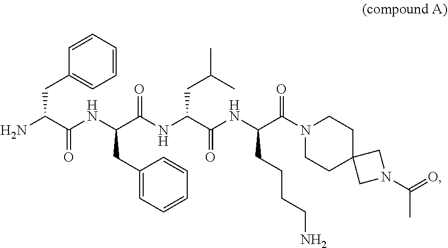 [SMILES: CC(=O)N1CC2(CCN(C(=O)[C@@H](CCCCN)NC(=O)[C@@H](CC(C)C)NC(=O)[C@@H](Cc3ccccc3)NC(=O)[C@H](N)Cc3ccccc3)CC2)C1]
and
b) an absorption enhancer.

2. The oral pharmaceutical composition of claim 1, wherein the absorption enhancer is selected from one or more of N-[8-(2-hydroxybenzoyl)amino]octanoic acid or a pharmaceutically acceptable salt thereof, 4-[(4-chloro-2-hydroxy-benzoyl)amino]butyric acid or a pharmaceutically acceptable salt thereof, lauroyl-L-carnitine or a hydrochloride thereof, sodium caprylate, sodium caprate, capric acid, caprylocaproyl macrogolglyceride.

3. The oral pharmaceutical composition of claim 2, wherein the absorption enhancer is N-[8 (2 hydroxybenzoyl)amino]octanoic acid or a pharmaceutically acceptable salt thereof.

4. The oral pharmaceutical composition of claim 3, wherein the weight ratio of compound A to N-[8-(2-hydroxybenzoyl)amino]octanoic acid or a pharmaceutically acceptable salt thereof is 1:20 to 1:80.

5. (canceled)

6. The oral pharmaceutical composition of claim 2, wherein the absorption enhancer is 4-[(4-chloro-2-hydroxy-benzoyl)amino]butyric acid or a pharmaceutically acceptable salt thereof.

7. The oral pharmaceutical composition of claim 6, wherein the weight ratio of compound A to 4-[(4-chloro-2-hydroxy-benzoyl)amino]butyric acid or a pharmaceutically acceptable salt thereof is 1:20 to 1:200.

8. The oral pharmaceutical composition of claim 6, further comprising one or more non-active ingredients selected from
(1) a filler,
(2) a binder,
(3) a disintegrant,
(4) a pH regulator,
(5) a surfactant, and
(6) a lubricant.

9. The oral pharmaceutical composition of claim 8, wherein the non-active ingredient is a lubricant, and the weight ratio of compound A:4-[(4-chloro-2-hydroxy-benzoyl)amino]butyric acid or a pharmaceutically acceptable salt thereof:the lubricant is 1:(20-200):(0.2-2).

10. The oral pharmaceutical composition of claim 9, wherein the lubricant is magnesium stearate.

11. The oral pharmaceutical composition of claim 8, wherein the non-active ingredient is a filler and a binder, and the weight ratio of compound A:4-[(4-chloro-2-hydroxy-benzoyl)amino]butyric acid or a pharmaceutically acceptable salt thereof:the filler:the binder is 1:(20-200):200:5.

12. The oral pharmaceutical composition of claim 11, wherein the filler is microcrystalline cellulose, and the binder is povidone.

13. The oral pharmaceutical composition of claim 8, wherein the non-active ingredient is a filler, a binder and a lubricant, and the weight ratio of compound A:4-[(4-chloro-2-hydroxy-benzoyl)amino]butyric acid or a pharmaceutically acceptable salt thereof:the filler:the binder:the lubricant is 1:(20-200):200:5:(1-5).

14. The oral pharmaceutical composition of claim 13, wherein the filler is selected from microcrystalline cellulose and anhydrous calcium hydrogenphosphate, the binder is povidone, and the lubricant is magnesium stearate.

15. The oral pharmaceutical composition of claim 8, wherein the non-active ingredient is a surfactant, a filler and a lubricant, and the weight ratio of compound A:4-[(4-chloro-2-hydroxy-benzoyl)amino]butyric acid or a pharmaceutically acceptable salt thereof:the surfactant:the filler:the lubricant is 3:200:6:200:1.

16. The oral pharmaceutical composition of claim 15, wherein the surfactant is propylene glycol monolaurate and/or polyethylene glycol, the filler is anhydrous calcium hydrogenphosphate, and the lubricant is magnesium stearate.

17. The oral pharmaceutical composition of claim 2, wherein the absorption enhancer is lauroyl-L-carnitine or a hydrochloride thereof.

18. The oral pharmaceutical composition of claim 17, wherein the weight ratio of compound A to lauroyl-L-carnitine or a hydrochloride thereof is 1:10-1:150.

19. The oral pharmaceutical composition of claim 17, further comprising one or more non-active ingredients selected from
(1) a filler, (2) a binder (3) a disintegrant, (4) a pH regulator, (5) a surfactant, and (6) a lubricant.

20. The oral pharmaceutical composition of claim 19, wherein the non-active ingredient is a pH regulator; the weight ratio of compound A:lauroyl-L-carnitine or a hydrochloride thereof:the pH regulator is 1:(10-50):24.

21. The oral pharmaceutical composition of claim 20, wherein, the pH regulator is citric acid.

22. The oral pharmaceutical composition of claim 2, wherein the absorption enhancer is sodium caprate.

23. The oral pharmaceutical composition of claim 22, wherein the weight ratio of compound A to sodium caprate is 1:50-1:200.

24. (canceled)

25. The oral pharmaceutical composition of claim 2, wherein the absorption enhancer is capric acid.

26. (canceled)

27. The oral pharmaceutical composition of claim 2, wherein the absorption enhancer is caprylocaproyl macrogolglyceride.

28. The oral pharmaceutical composition of claim 27, wherein the weight ratio of compound A to caprylocaproyl macrogolglyceride is 1:600-1:3600.

29. (canceled)

30. A method for preparing the oral pharmaceutical composition of claim 1, comprising the following step: compound A, the absorption enhancer and other non-active ingredients are mixed directly, and then filled into capsules or compressed into tablets; or compound A, the absorption enhancer and other non-active ingredients are wet granulated and then filled into capsules or compressed into tablets; or compound A, the absorption enhancer and other hydrophilic non-active ingredients are wet granulated, then dispersed in a hydrophobic medium, and filled into capsules; or compound A, the absorption enhancer and other non-active ingredients are dissolved in purified water to prepare a solution; or compound A, the absorption enhancer and other non-active ingredients are dissolved, dried and pulverized, and then filled into capsules or compressed into tablets.

31. A method for treating a disease or a condition related to κ-opioid receptor, comprising administering an oral pharmaceutical composition of claim 1.

32. (canceled)

33. (canceled)

34. (canceled)
